(19) United States
(12) Patent Application Publication
(10) Pub. No.: US 2008/0119697 A1
(43) Pub. Date: May 22, 2008

(54) BIDIRECTIONAL COMMUNICATION INTERFACE

(75) Inventors: Sachin Vadodaria, Fox Point, WI (US); Claudio Patricio Mejia, Wauwatosa, WI (US); Richard William Schefelker, Menomonee Falls, WI (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/561,660

(22) Filed: Nov. 20, 2006

(51) Int. Cl. A61B 5/00

(52) U.S. Cl. 600/301

(57) ABSTRACT

A communication module is presented. The communication module includes a communication interface operationally coupled to a monitoring system and a mapping system, where the communication interface is configured to facilitate bidirectional communication of data between the monitoring system and the mapping system. A method and computer-readable medium that afford functionality of the type defined by this communication module are also contemplated in conjunction with the present technique.

### BACKGROUND

The invention relates generally to diagnostic systems, and more particularly to integration of data between various devices in the diagnostic systems.

Diagnostic analysis has emerged into an essential aspect of patient care in fields, such as clinical interventional procedures such as interventional cardiology that include cardiac electrophysiology or cardiac angiography, for instance. For example, in areas such as interventional cardiology, various systems, such as, but not limited to, a monitoring and recording system and a mapping and localization system, may be employed to facilitate the interventional procedures.

During cardiac interventional procedures, probes, such as multi-polar catheters, are positioned inside the anatomy, such as the heart, and electrical recordings are made from the different chambers of the heart. These catheters are typically inserted into a vein, such as the femoral vein, and guided to the heart through the vasculature of the patient. Data is acquired via these catheters by a system such as the monitoring system. As will be appreciated, a large amount of data is generally collected during the interventional procedure. The acquired data is then analyzed to aid a clinician in the diagnosis of physiological problems and determination of appropriate treatment options. Additionally, another system, such as the mapping system is used to create graphical displays of cardiac structures to aid in the identification, characterization and localization of physiological problems.

A drawback of the currently available techniques however is that these procedures are extremely tedious requiring considerable manpower, time and expense as an inordinate amount of time is spent in collecting and analyzing the data. More particularly, use of currently available monitoring and mapping systems entails collection of data by two separate systems and by at least two independent clinicians. Presently, the data is manually acquired at both the monitoring and the mapping systems. The two sets of data are then manually collated and transmitted to a data storage system, such as a hospital information system (HIS). Additionally, clinicians conducting cardiac electrophysiological studies typically work with physically separate and electronically isolated systems for cardiac monitoring and mapping in order to assess the electrical properties of the heart muscle within the anatomy of the heart while continuously monitoring the position of one or more catheters disposed within the anatomy of the patient including the heart. In other words, use of the currently available systems requires the clinicians to shuttle back and forth between two or more workstations, as the clinicians are unable to simultaneously visualize the different sets of data at a single, centralized location. These tedious processes disadvantageously detract from the interventional procedure and result in diminished procedural efficiency. Consequently, the currently available techniques impede the workflow thereby interfering with a caregiver providing timely critical care to the patient.

There is therefore a need for a design that permits simultaneous real-time centralized access to the different sets of data on a single system for recording and analysis during an interventional procedure. In particular, there is a significant need for a design of an interface configured to facilitate multi-directional communication between the various devices involved in the diagnostic system, thereby resulting in enhanced workflow efficiencies in a caregiving facility and enhanced patient care. Additionally, it may be desirable to develop a technique that coalesces the multiple sets of data to generate a convenient, single consolidated case report form.

### BRIEF DESCRIPTION

In accordance with aspects of the present technique, a communication module is presented. The communication module includes a communication interface operationally coupled to a monitoring system and a mapping system, where the communication interface is configured to facilitate bidirectional communication of data between the monitoring system and the mapping system.

In accordance with another aspect of the present technique, a method for imaging is presented. The method includes outputting in real-time a first set of data and a second set of data for display on a single display unit of an imaging system, where the imaging system comprises at least a monitoring system and a mapping system. Computer-readable medium that afford functionality of the type defined by this method is also contemplated in conjunction with the present technique.

In accordance with further aspects of the present technique a system for imaging is presented. The system includes a monitoring system. Further, the system also includes a mapping system. Additionally, the system includes a communication module operationally coupled to the monitoring system and the mapping system, where the communication module comprises a communication interface configured to facilitate bidirectional communication of data between the monitoring system and the mapping system.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an exemplary diagnostic system, in accordance with aspects of the present technique;

FIG. 2 is a front view of an imaging system of the exemplary diagnostic system of FIG. 1, in accordance with aspects of the present technique;

FIG. 3 is a front view of a monitoring system component illustrating an exemplary process of data communication between a monitoring system component and a mapping system component of the imaging system of FIG. 2, in accordance with aspects of the present technique;

FIG. 4 is a front view of a mapping system component illustrating an exemplary process of data communication between a mapping system component and a monitoring system component of the imaging system of FIG. 2, in accordance with aspects of the present technique; and

FIG. 5 is a flow chart illustrating an exemplary process of bidirectional data communication for imaging, in accordance with aspects of the present technique.

### DETAILED DESCRIPTION

As will be described in detail hereinafter, an exemplary diagnostic system and method in accordance with exemplary aspects of the present technique are presented. During an interventional procedure where one or more catheters are employed for monitoring and/or treatment, it is desirable to visualize different sets of data on a single, centralized location to aid the clinician guide the catheters to a desirable destination within the vasculature of the patient and/or deliver therapy.

Although, the exemplary embodiments illustrated hereinafter are described in the context of a medical imaging system, it will be appreciated that use of the diagnostic system in industrial applications are also contemplated in conjunction with the present technique.

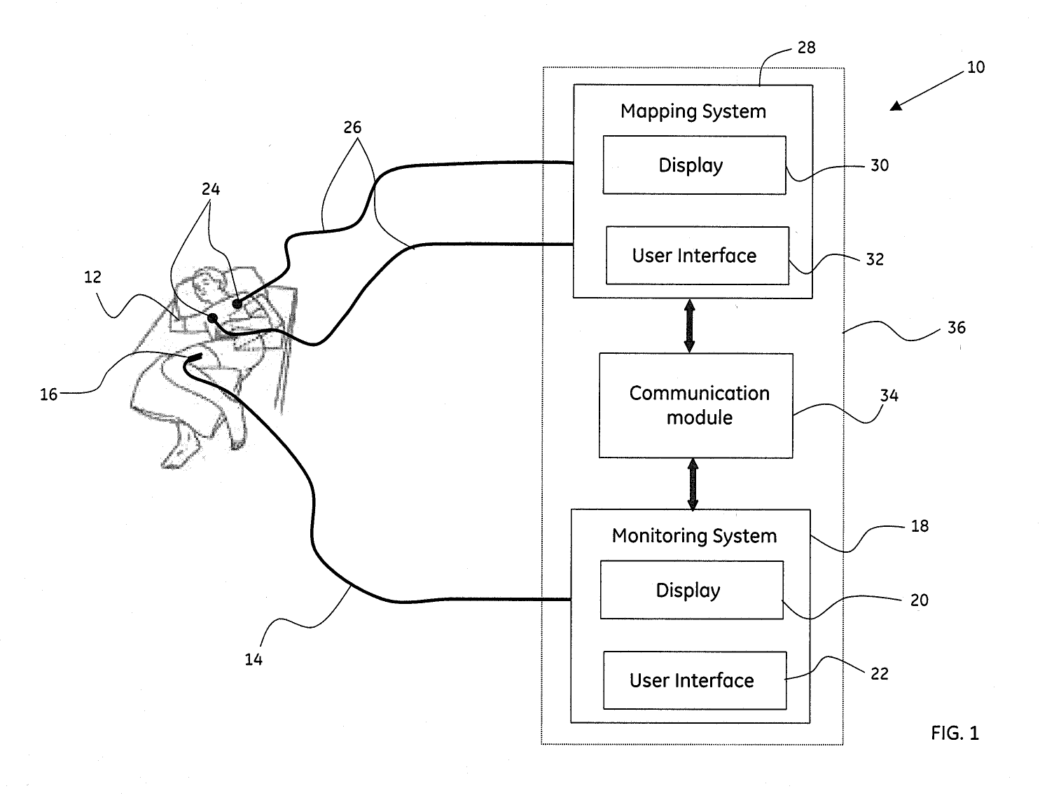

FIG. 1 is a block diagram of an exemplary diagnostic system 10 for use in patient monitoring and treatment in accordance with aspects of the present technique. In a presently contemplated configuration, the diagnostic system 10 may be configured to facilitate acquisition of physiological data from a patient 12 via a probe 14. It may be noted that the physiological data may include vital signs, such as, but not limited to, a blood pressure, a temperature, a blood oxygen level, or an electrocardiogram, or a combination thereof. In accordance with aspects of the present technique, the probe 14 may be configured to facilitate interventional procedures. It should also be noted that although the embodiments illustrated are described in the context of a catheter-based probe, other types of probes such as endoscopes, laparoscopes, surgical probes, probes adapted for interventional procedures, or combinations thereof are also contemplated in conjunction with the present technique. Reference numeral 16 is representative of a portion of the probe 14 disposed inside the vasculature of the patient 12.

In addition, an external probe, such as an external ultrasound probe may also be employed to aid in the acquisition of physiological data. Also, one or more sensors (not shown) may be disposed on the patient 12 to assist in the acquisition of physiological data. These sensors may be operationally coupled to a data acquisition device via leads (not shown), for example.

The system 10 may also include a monitoring and recording system 18 that is in operative association with the probe 14 and configured to facilitate acquisition of physiological data from the patient 12 via the probe 14 and/or sensors disposed on the patient 12. It may be noted that the terms monitoring and recording system and monitoring system may be used interchangeably. As will be appreciated, the physiological monitoring and recording system 18 may be configured to closely monitor the electrical function of the patient's heart and facilitate evaluation of heart rhythms that will in turn facilitate a clinician to determine an appropriate treatment option, for example.

In certain embodiments, the monitoring system 18 may include an electrophysiological monitoring system. Alternatively, the monitoring system 18 may include a hemodynamic monitoring system. Also, a combination of an electrophysiological monitoring system and a hemodynamic monitoring system may be employed as the monitoring system 18. Further, physiological data acquired via the monitoring system 18 may include a blood pressure, a temperature, a blood oxygen level, or an electrocardiogram, as previously noted. It should be noted that although the exemplary embodiments illustrated hereinafter are described in the context of a medical imaging system, such as, but not limited to, ultrasound imaging systems, optical imaging systems, computed tomography (CT) imaging systems, magnetic resonance (MR) imaging systems, X-ray imaging systems, or positron emission tomography (PET) imaging systems, other imaging systems, such as, but not limited to, pipeline inspection systems, liquid reactor inspection systems, or other imaging systems are also contemplated in accordance with aspects of the present technique.

Further, the monitoring system 18 may also be configured to generate a graphical representation, for example, of the acquired physiological data for presentation on a display. As illustrated in FIG. 1, the monitoring system 18 may include a display 20 and a user interface 22. In accordance with aspects of the present technique, the display 20 of the monitoring system 18 may be configured to aid in the visualization of the physiological data acquired by the monitoring system 18. More particularly, the display 20 may be configured to aid a clinician in visualizing and monitoring the vital signs of the patient 12. Although, the exemplary embodiment illustrated in FIG. 1 is shown as including one display 20, it will be appreciated that use of more than one display is also contemplated in conjunction with the present technique.

Additionally, the user interface 22 of the monitoring system 18 may include a human interface device (not shown) configured to facilitate the clinician to manipulate the acquisition and/or visualization of the physiological data acquired from the patient 12. The human interface device may include a mouse-type device, a trackball, a joystick, or a stylus. However, as will be appreciated, other human interface devices, such as, but not limited to, a touch screen, may also be employed.

As will be appreciated, one or more probes (not shown) that may be configured to image one or more anatomical regions may be disposed within the anatomy of the patient 12. The images of these anatomical regions may then be employed to facilitate assessing need for therapy in the one or more regions of interest within the anatomical regions. Additionally, in certain embodiments the probes may also be configured to facilitate delivery of therapy to the identified one or more regions of interest within the anatomy of the patient 12. As used herein, “therapy” is representative of ablation, percutaneous ethanol injection (PEI), cryotherapy, and laser-induced thermotherapy. Further, “therapy” may also include delivery of tools, such as needles for delivering gene therapy, for example. Also, as used herein, “delivering” may include various means of providing therapy to the one or more regions of interest, such as conveying therapy to the one or more regions of interest or directing therapy towards the one or more regions of interest. As will be appreciated, in certain embodiments the delivery of therapy, such as RF ablation, may necessitate physical contact with the one or more regions of interest requiring therapy. However, in certain other embodiments, the delivery of therapy, such as high intensity focused ultrasound (HIFU) energy, may not require physical contact with the one or more regions of interest requiring therapy.

During an electrophysiological procedure, such as invasive cardiology, one or more probes may be disposed within the anatomy of the patient 12 to aid in imaging and/or delivery of therapy to one or more regions of interest, as noted hereinabove. Accordingly, the system 10 may also include a mapping and localization system 28 that is in operative association with the patient 12 and configured to facilitate acquisition of mapping data from the patient 12 via one or more sensors 24 disposed on the patient 12. Further, the mapping system 28 may be operatively coupled to the sensors 24 on the patient via leads 26. It may be noted that the terms mapping and localization system and mapping system may be used interchangeably.

As will be appreciated, the mapping and localization system 28 has grown to serve as a tool for facilitating electrophysiology procedures. More particularly, the mapping system 28 may be advantageously configured to aid in the process of identification, characterization and localization of regions of interest. The mapping system 28 may also be configured to assist in obtaining localization coordinates, such as XYZ coordinates, of the one or more probes disposed within the vasculature of the patient 12. Data associated with the identification, characterization and localization of regions of interest may be collectively referred to as mapping data. For example, mapping data may include voltage, time, thermal data, acoustic data, or localization coordinates, or a combination thereof. Furthermore, the mapping system 28 may also be configured to monitor the progression of the probes within the vasculature of the patient 12. Accordingly, the localization coordinates and progression of the probes within the vasculature of the patient 12 may be visualized by displaying the mapping data on a portion of a display of the mapping system 28. In other words, the mapping system 28 may be configured to create three-dimensional graphical displays of cardiac structures and arrhythmias, and also enable localization and navigation of the probes without the use of fluoroscopy. In a presently contemplated configuration, the mapping system 28 may include a three-dimensional mapping system. However, as will be appreciated use of other mapping systems is also envisaged in accordance with aspects of the present technique. The mapping system 28 may include a catheter-based mapping system, a contact-based mapping system, or a combination thereof.

In one embodiment, the mapping system 28 may include a display 30 and a user interface 32, as illustrated in FIG. 1. As previously noted with reference to the monitoring system 18, the display 30 of the mapping system 28 may be configured to aid in the visualization of the mapping data acquired by the mapping system 28. Although, the embodiment illustrated in FIG. 1 depicts the mapping system 28 as including one display 30, it will be appreciated that use of more than one display is also contemplated in conjunction with the present technique.

Further, the user interface 32 of the mapping system 28 may include a human interface device (not shown) configured to facilitate the user in identifying the one or more regions of interest and/or the acquisition of information associated with the location of the one or more probes using the image of the anatomical region displayed on the display 30, as previously described with reference to the monitoring system 18.

Although the connections between the patient 12 and the monitoring system 18 and the mapping system 28 are illustrated as being wired connections, it will be appreciated that wireless connections may also be used to facilitate acquisition of physiological and/or mapping data from the patient 12.

As previously noted, clinicians conducting electrophysiological studies employing the presently available diagnostic systems typically work with physically separate and electronically isolated systems for cardiac monitoring and mapping. In addition, using the currently available systems, the clinicians are unable to simultaneously visualize the different sets of data at a single centralized location, thereby resulting in tedious processes that disadvantageously detract from the interventional procedure and result in diminished procedural efficiency. There is therefore a need for a design that facilitates simultaneous real-time centralized access to the different sets of data on a single system for recording and analysis.

Accordingly, an exemplary communication module 34 that may be configured to facilitate bidirectional communication of data between the monitoring system 18 and the mapping system 28 is presented. In accordance with aspects of the present technique, the communication module 34 may include a communication interface (not shown) that is operationally coupled to the monitoring system 18 and the mapping system 28. Furthermore, in certain embodiments, the communication interface may include a wired interface, a wireless interface, an Ethernet interface, a Bluetooth interface, or a combination thereof.

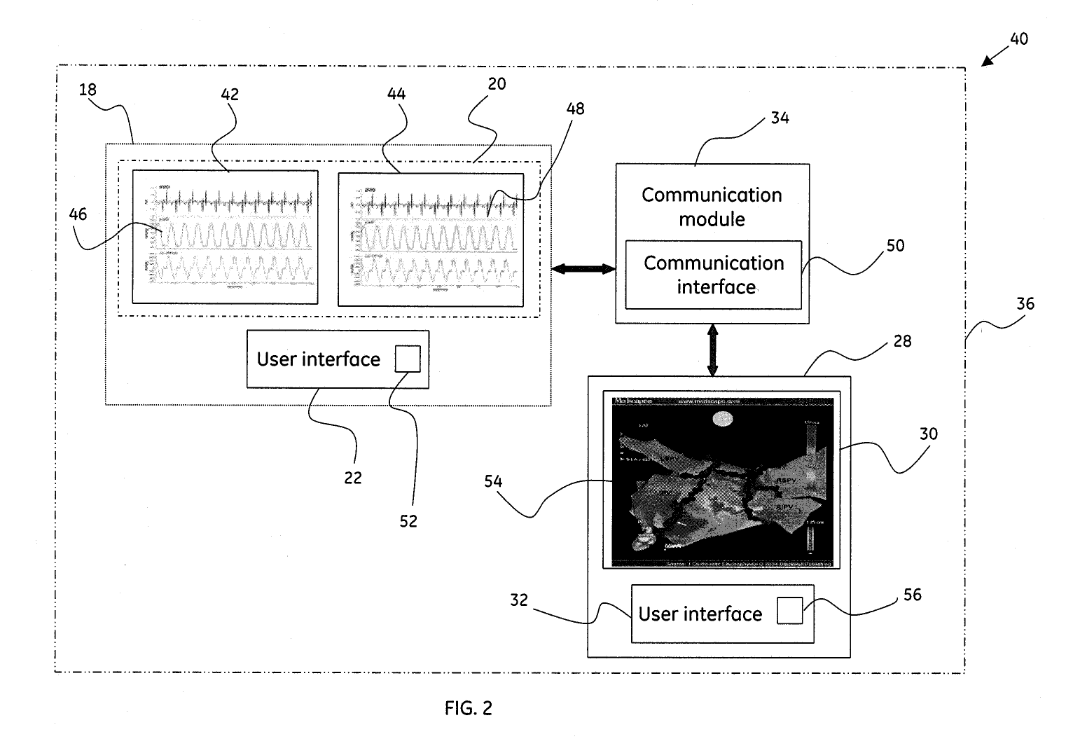

The communication interface may be configured to facilitate communication of data from the monitoring system 18 to the mapping system 28, in certain embodiments. Further, the communication interface may also be configured to aid in the communication of data from the mapping system 28 to the monitoring system 18, in certain other embodiments. Additionally, bidirectional communication of data between the monitoring system 18 and the mapping system 28 may also be facilitated by the communication interface. In particular, the communication interface may be configured to facilitate communication of physiological data from the monitoring system 18 to the mapping system 28 and the communication of mapping data from the mapping system 28 to the monitoring system 18. The exemplary process of bidirectional communication of data between the monitoring system 18 and the mapping system 28 will be described in greater detail with reference to FIGS. 2-5. Also, the monitoring system 18, the mapping system 28 and the communication module 34 may be collectively referred to as an imaging system and may be generally represented by reference numeral 36.

Turning now to FIG. 2, a front view 40 of the imaging system 36 (see FIG. 1) of the exemplary diagnostic system 10 of FIG. 1 is illustrated. As previously noted with reference to FIG. 1, the monitoring system 18 may include one or more displays. In a presently contemplated configuration, the display 20 of the monitoring system 18 is shown as having a first display 42 and a second display 44. Physiological data acquired by the monitoring system 18 and displayed on the first display 42 may be generally represented by reference numeral 46, while reference numeral 48 is representative of physiological data acquired by the monitoring system 18 and displayed on the second display 44. It may be noted that physiological data 46, 48 displayed on the first and second displays 42, 44 respectively may embody the same set of data. Alternatively, reference numerals 46, 48 may correspond to two different sets of physiological data. Further, in the illustrated embodiment of FIG. 2, the mapping system 28 is shown as including one display 30. Also, reference numeral 54 embodies mapping data acquired by the mapping system 28 and displayed on the display 30 of the mapping system 28.

As will be appreciated, the physiological data 46, 48 is displayed on the displays 42, 44 of the monitoring system 18, while mapping data 54 is displayed on the display 30 of the mapping system 28. Consequently, clinicians conducting electrophysiological studies need to work with the physically separate and electronically isolated monitoring and mapping systems 18, 28 as the clinicians are unable to simultaneously visualize the different sets of data at a single, centralized location. Accordingly, as previously noted, an exemplary communication interface configured to facilitate simultaneous real-time centralized access to the different sets of data on a single system is presented.

The communication module 34 may include a communication interface 50 configured to facilitate the bidirectional communication of data between the monitoring system 18 and the mapping system 28, as previously noted. Further, the communication interface 50 may include a hardware component, a software component, or both. For example, the hardware component may include a computer, a monitor, or a keyboard, to name a few, while the software component may include software applications, such as software modules associated with the monitoring system 18, the mapping system 28 and the communication interface 50. It may be noted that communication protocols, such as, but not limited to Ethernet based communication protocols, that are compatible to both the monitoring system 18 and the mapping system 28 may be employed to facilitate bidirectional communication between the two systems 18, 28.

In accordance with exemplary aspects of the present technique, the communication interface 50 is configured to facilitate bidirectional interaction between the monitoring system 18 and the mapping system 28. It may be noted that, in certain embodiments, the communication interface 50 may be configured to allow the monitoring system 18 and the mapping system 28 to operate independent of one another. In other words, each of the monitoring system 18 and the mapping system 28 uses the respective functionality.

Alternatively, the communication interface 50 may be configured to operate in an “interfaced” mode. As used herein, “interfaced” mode embodies a mode of operating the imaging system 36, where the monitoring system 18 and the mapping system 28 are in operative communication with one another. Furthermore, the imaging system 36 may be operated in the interfaced mode in response to a trigger signal. This trigger signal may be generated in response to the clinician selecting the interfaced mode of operating the imaging system 36. In a presently contemplated configuration, the trigger signal may be generated in response to the clinician choosing the interfaced mode of operation by selecting an icon 52 on the user interface 22 of the monitoring system 18. Alternatively, the clinician may also initiate the generation of the trigger signal by selecting an icon 56 on the user interface 32 of the mapping system 28, thereby activating the interfaced mode of operation of the imaging system 36. Furthermore, the clinician may activate the interfaced mode of operating the imaging system 36 by selecting both the icons 52, 56. In accordance with further aspects of the present technique, icons representative of operating the imaging system 36 in the interfaced mode may also be disposed on the displays 42, 44 of the monitoring system 18 and/or on the display 30 of the mapping system 28.

As previously noted, use of currently available techniques entails manual entry of patient demographic data at both the monitoring system 18 and the mapping system 28, typically by more than one clinician, thereby resulting in the duplication of data. However, in accordance with aspects of the present technique, the imaging system 36 may be configured to facilitate entry of patient demographic data either at the monitoring system 18 or the mapping system 28, thereby reducing duplication of data entry and advantageously enhancing workflow. As will be appreciated, patient demographic data may include name of patient, vital statistics, date of birth, social security number, and medical record number, to name a few. The patient demographic data may then be communicated to other of the mapping system 28 or the monitoring system 18.

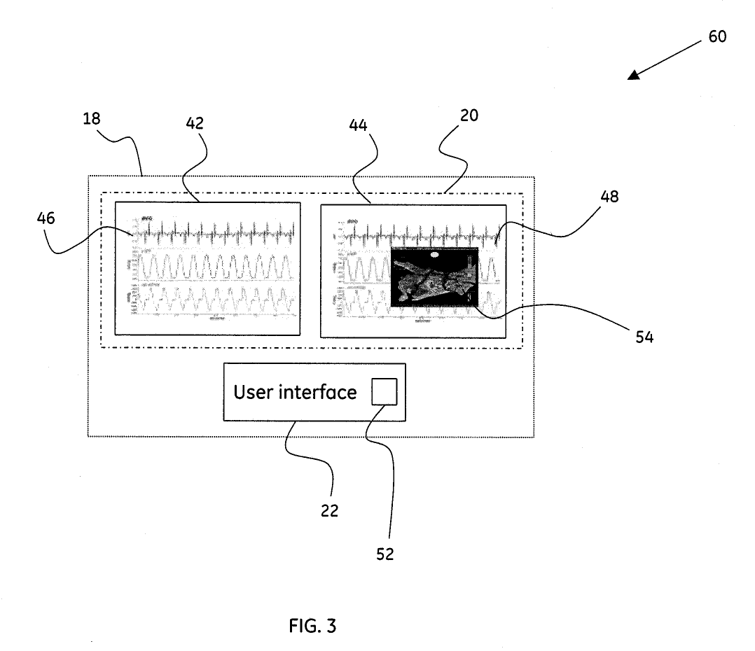

In the interfaced mode of operation, the imaging system 36 may be configured to allow bidirectional communication of data between the monitoring system 18 and the mapping system 28, as previously described. More particularly, in response to receipt of the trigger signal, data such as mapping data may be communicated from the mapping system 28 to the monitoring system 18. The mapping data may include voltage, time, thermal data, acoustic data or localization coordinates, as previously noted. Further, the mapping data so communicated to the monitoring system 18 may be overlaid in real-time on a portion of the display 20 of the monitoring system 18, as illustrated in FIG. 3.

FIG. 3 is a front view 60 of the monitoring system 18 illustrating an exemplary process of data communication between the monitoring system 18 (see FIGS. 1-2) and the mapping system 28 (see FIGS. 1-2). In the illustrated embodiment, mapping data 54 (see FIG. 2) from the mapping system 28 is shown as being overlaid on a portion of the second display 44 of the monitoring system 18. In a presently contemplated configuration, the physiological data 48 and the mapping data 54 may be displayed in separate windows. These windows may be overlaid on one another on a single display, such as the second display 44. However, as will be appreciated, the mapping data 54 may also be overlaid on a portion of the first display 42 of the monitoring system 18. It may be noted that, in certain other embodiments, the mapping data 54 may be overlaid on the physiological data 46 in a predetermined region of the first display 42, or on the physiological data 48 in a predetermined region of the second display 44, or both. Consequently, overlaying the mapping data 54 on the physiological data 48 on a single display 44, for example, advantageously enables the clinician to view both the physiological data 48 and mapping data 54 simultaneously and in real-time, thereby greatly enhancing identification of physiological problems, if any, and determination of physical locations of the physiological problems.

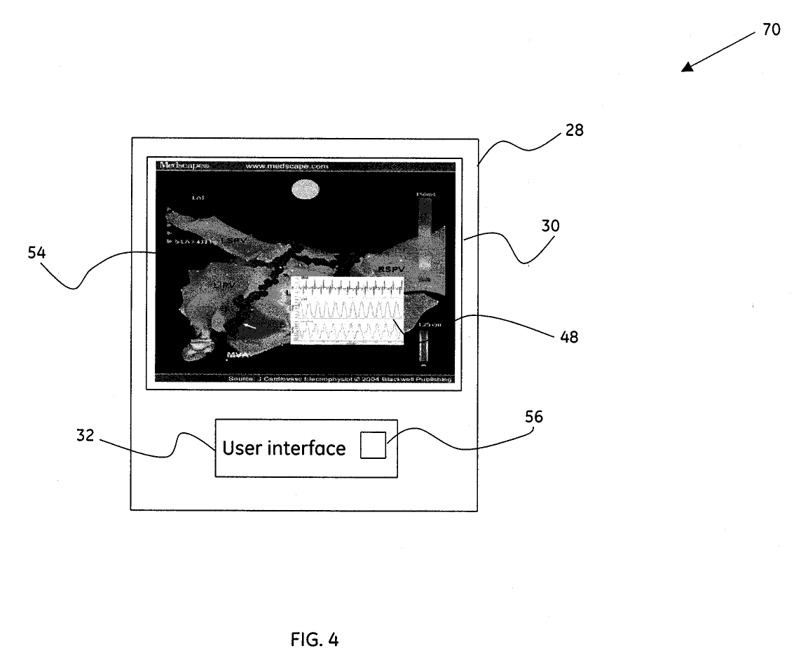

With returning reference to FIG. 2, in certain other embodiments, physiological data 46, 48 from the monitoring system 18 may be communicated in real-time to the mapping system 28 in response to a trigger signal. Physiological data may include a blood pressure, a temperature, a blood oxygen level, or an echocardiogram, as previously described. The physiological data so communicated to the mapping system 28 may be overlaid in real-time on a portion of the display 30 of the mapping system 28 as illustrated in FIG. 4.

Referring now to FIG. 4, a front view 70 of the mapping system 28 depicting an exemplary process of data communication between the mapping system 28 (see FIGS. 1-2) and the monitoring system 18 (see FIGS. 1-2) is illustrated. In the illustrated embodiment, physiological data, such as physiological data 48 (see FIG. 2) from the monitoring system 18, for example, may be overlaid on a portion of the display 30 of the mapping system 28. Furthermore, it may be noted that physiological data 48 may be overlaid on the mapping data 54 in a predetermined region of the display 30 of the mapping system 28. As previously noted with reference to FIG. 3, the physiological data 48 and the mapping data 54 may be displayed in separate windows and overlaid on one another on a single display, such as the display 30. Here again, overlaying the physiological data 48 on the mapping data 54 on a single display 30 allows the clinician to simultaneously view both the physiological data 48 and mapping data 54 in real-time, thereby greatly enhancing identification of physiological problems, if any, and determination of physical locations of the physiological problems.

With returning reference to FIG. 2, the physiological data 46, 48 from the monitoring system 18 and the mapping data 54 from the mapping system 28 may be coalesced to generate a consolidated report for display on the monitoring system 18, the mapping system 28, or both. More particularly, as illustrated in FIG. 3, the mapping data 54 that has been overlaid on the physiological data 48 may be combined to generate a single consolidated report for display on the monitoring system 18. In one embodiment, the consolidated report may include a consolidated image representative of both the physiological data 48 and the mapping data 54. This consolidated image may be configured to provide a larger context to aid in the visualization of patient data. Similarly, the physiological data 48 that has been overlaid on the mapping data 54 may be coalesced to generate a consolidated report for display on the mapping system 28. However, in certain other embodiments, the consolidated report may be displayed on both the monitoring system 18 and the mapping system 28. Additionally, the consolidated report so generated may be recorded and/or transmitted for storage. For example, the consolidated report may be transmitted to a HIS or a doctor's office for storage and/or further analysis.

By implementing the exemplary communication interface 50 as described hereinabove, efficiency of workflow may be greatly enhanced. More particularly, use of the communication interface 50 facilitates both the monitoring system 18 and the mapping system 28 to utilize respective current functionalities. Additionally, the communication interface 50 may be configured to facilitate real-time centralized data management on one of the monitoring system 18 or the mapping system 28, or both, thereby enabling the clinician to proceed through the clinical procedure at a faster pace as the clinician is able to simultaneously view in real-time both the physiological data and mapping data on a single display. Also, the physiological data and the mapping data may be displayed in multiple windows of a single display or in multiple windows of multiple displays to facilitate efficient identification of physiological problems and determination of physical locations of the physiological problems to aid in formulation of possible therapies. Moreover, use of the communication interface 50 eliminates need for duplication of data entry, such as patient demographic data, in the monitoring system 18 and the mapping system 28, thereby resulting in enhanced efficiency of the clinical procedure.

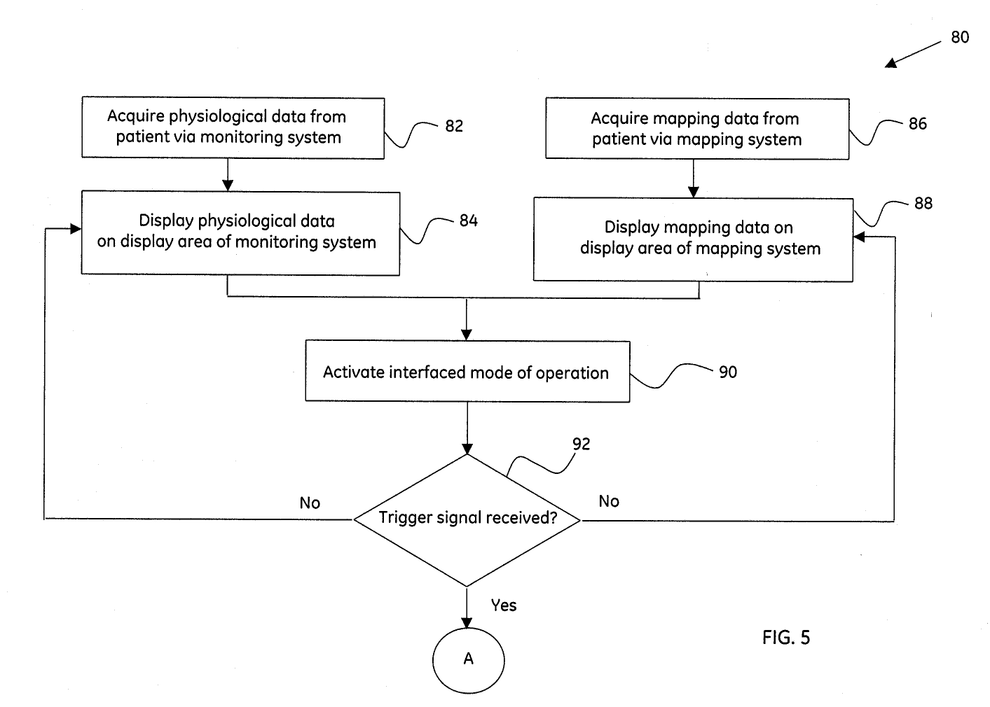

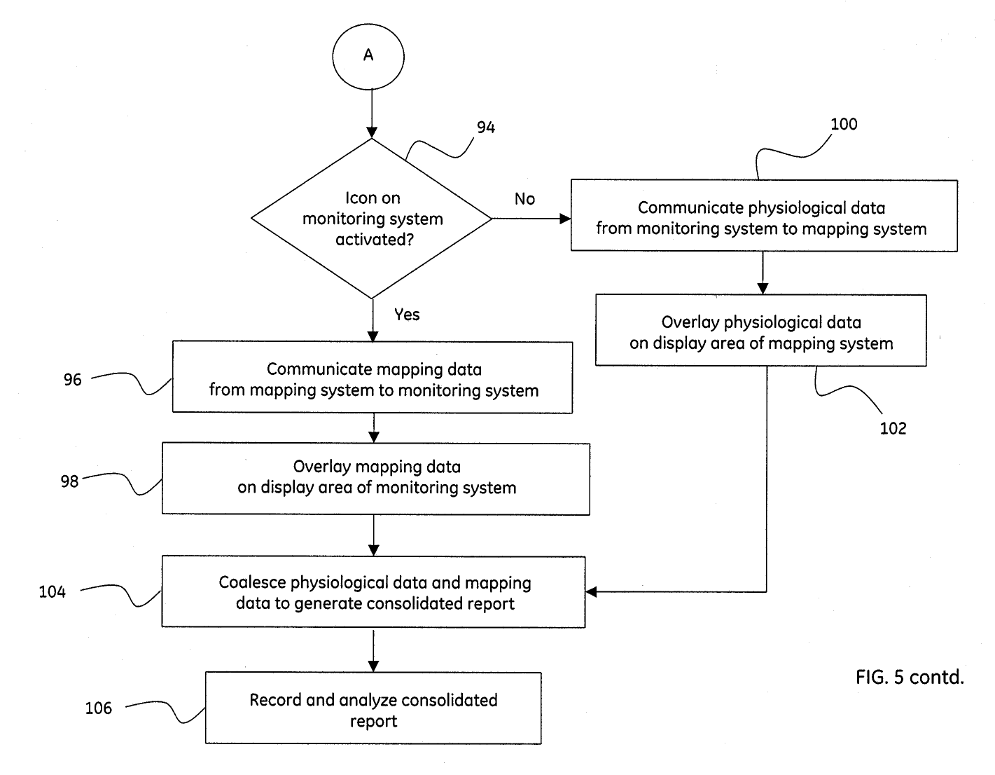

FIG. 5 is a flow chart 80 illustrating an exemplary process of bidirectional data communication for imaging. In accordance with exemplary aspects of the present technique, a method for imaging is presented. The method starts at step 82 where physiological data representative of the patient 12 (see FIG. 1) may be acquired by the monitoring system 18 (see FIG. 1) via a probe, such as the imaging catheter 14 (see FIG. 1), for example. The physiological data may be acquired in real-time employing the imaging catheter 14 and/or sensors disposed on the patient 12. Further, at step 84, the physiological data so acquired may be displayed on a display of the monitoring system 18 to aid the user in visualizing the physiological data. It should be noted that mechanical means, electronic means, or combinations thereof may be employed to facilitate the acquisition of the physiological data. Alternatively, previously stored physiological data representative of the patient 12 may be acquired by the monitoring system 18.

In a similar fashion, at step 86, mapping data may also be acquired from the patient 12 by the mapping system, such as the mapping system 28 (see FIG. 1). Further, at step 88, the mapping data may be displayed on the mapping system 28 to assist the user in visualizing the region of interest being imaged. In one embodiment, steps 82-84 may be carried out concurrently with steps 86-88.

As previously described, the imaging system 36 (see FIG. 1) may be configured to operate in the interfaced mode in response to a trigger signal, where the monitoring system 18 is in bidirectional communication with the mapping system 28. Accordingly, the clinician may activate the interfaced mode of operating the imaging system 36 by activating the icon 52 (see FIG. 2) on the monitoring system 18, or the icon 56 (see FIG. 2) on the mapping system 28, or both, as indicated by step 90. Furthermore, the imaging system 36 may be configured to generate the trigger signal in response to the clinician activating the icon 52 on the monitoring system 18 or the icon 56 on the mapping system, or both. In one embodiment, the trigger signal may be in an OFF state when the monitoring system 18 and the mapping system 28 are operating in the independent mode. The status of the trigger signal may be transitioned to an ON state when the mode of operating the monitoring system 18 and the mapping system 28 is switched to the interfaced mode.

Subsequently, at step 92, a check is carried out to verify if the trigger signal has been received. In other words, a check is carried out to verify if the status of trigger signal has been transitioned from the OFF state to the ON state. Following step 92, if no change in the status of the trigger signal is detected, the imaging system 36 may be configured to continue operating in the independent mode. However, if the imaging system 36 detects a change in status of the trigger signal from the OFF state to the ON state, a further check may be carried out at step 94 to identify the source of the trigger signal. In other words, at step 94, a check is carried out to verify if the icon 52 on the monitoring system 18 is activated. If the icon 52 is activated, mapping data may be communicated in real-time from the mapping system 28 to the monitoring system 18 at step 96. Following step 96, the mapping data may be overlaid on the physiological data on a predetermined region of the display of the monitoring system 18, at step 98. In other words, at step 98, an updated image including both the physiological data and the mapping data may be generated and displayed on a display, such as the second display 44 of the monitoring system 18.

With returning reference to the decision block 94, if icon 56 on the mapping system 28 is activated, physiological data from the monitoring system 18 may be communicated in real-time to the mapping system 28, at step 100. Subsequently, at step 102, the physiological data may be overlaid on the mapping data on a predetermined region of the display of the mapping system 28. Here again, an updated image including both the physiological data and the mapping data may be generated and displayed on a display, such as the display 30 of the mapping system 28.

Following steps 98 and 102, the user may visualize an image including both the physiological data and the mapping data displayed on the display of either the monitoring system 18, the mapping system 28, or both, as previously noted. Consequently, the clinician may visualize the image representative of both the physiological data and the mapping data that is conveniently displayed on a single display. The clinician may then employ the image on the single display to identify physiological problems, and determine locations of the physiological problems. Additionally, the clinician may use the image to either monitor the locations of the one or more probes disposed within the anatomy of the patient 12 or guide one or more probes to a desirable location to facilitate imaging and/or delivery of therapy.

Subsequently, at step 104, images representative of the physiological data and the mapping data may be coalesced to generate a single consolidated report, as previously described. The consolidated report may then be transmitted to a storage facility, such as the HIS. Alternatively, the consolidated report may be recorded and analyzed by a clinician as indicated by step 106.

As will be appreciated by those of ordinary skill in the art, the foregoing example, demonstrations, and process steps may be implemented by suitable code on a processor-based system, such as a general-purpose or special-purpose computer. It should also be noted that different implementations of the present technique may perform some or all of the steps described herein in different orders or substantially concurrently, that is, in parallel. Furthermore, the functions may be implemented in a variety of programming languages, such as C++ or Java. Such code, as will be appreciated by those of ordinary skill in the art, may be stored or adapted for storage on one or more tangible, machine readable media, such as on memory chips, local or remote hard disks, optical disks (that is, CDs or DVDs), or other media, which may be accessed by a processor-based system to execute the stored code. Note that the tangible media may comprise paper or another suitable medium upon which the instructions are printed. For instance, the instructions can be electronically captured via optical scanning of the paper or other medium, then compiled, interpreted or otherwise processed in a suitable manner if necessary, and then stored in a computer memory.

The communication module, the system for imaging, and the method of imaging described hereinabove dramatically enhance efficiency of a clinical procedure that involves monitoring of different sets of data, such as physiological data and mapping data, acquired from the patient. Furthermore, an image representative of the physiological data as well as the mapping data is simultaneously displayed on a single display in real-time, thereby advantageously aiding the clinician in visualizing the current locations of the probes with respect to anatomical landmarks and subsequently in guiding the probes to desirable anatomical destinations to image and/or deliver therapy. Additionally, employing the techniques described hereinabove facilitates creation of a single, consolidated clinical report that advantageously includes procedural data acquired by both the monitoring system and the mapping system. Also, ease of use of the imaging system is substantially enhanced as the system entails entry of patient demographic data at only one of the monitoring system or the mapping system.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A communication module, comprising:
a communication interface operationally coupled to a monitoring system and a mapping system, wherein the communication interface is configured to facilitate bidirectional communication of data between the monitoring system and the mapping system.

2. The module of claim 1, wherein the communication interface is configured to facilitate communication of physiological data from the monitoring system to the mapping system or facilitate communication of mapping data from the mapping system to the monitoring system, or both.

3. The module of claim 2, wherein the physiological data comprises at least one of a temperature, a blood pressure, an electrocardiogram, or a blood oxygen level.

4. The module of claim 2, wherein the communication interface is configured to:
communicate the mapping data from the mapping system to the monitoring system; and
overlay the mapping data on a display of the monitoring system.

5. The module of claim 2, wherein the communication interface is configured to:
communicate the physiological data from the monitoring system to the mapping system; and
overlay the physiological data on a display of the mapping system.

6. The module of claim 2, wherein the communication interface is further configured to:
coalesce the physiological data from the monitoring system and the mapping data from the mapping system to generate a consolidated report for display on the monitoring system, the mapping system, or both.

7. The module of claim 1, wherein the communication interface comprises a wired interface, an Ethernet interface, a wireless interface, a Bluetooth interface, or a combination thereof.

8. The module of claim 1, wherein the monitoring system comprises an electrophysiological monitoring system, a hemodynamic monitoring system, or a combination thereof.

9. The module of claim 1, wherein the mapping system comprises a catheter-based mapping system, a contact-based mapping system, or a combination thereof.

10. A method for imaging, the method comprising:
outputting in real-time a first set of data and a second set of data for display on a single display unit of an imaging system, wherein the imaging system comprises at least a monitoring system and a mapping system.

11. The method of claim 10, wherein the first set of data comprises physiological data acquired via the monitoring system and the second set of data comprises mapping data acquired via the mapping system.

12. The method of claim 10, further comprising outputting in real-time the first set of data and the second set of data in response to a trigger.

13. The method of claim 10, further comprising:
communicating the second set of data from the mapping system to the monitoring system; and
overlaying the second set of data on a display of the monitoring system.

14. The method of claim 10, further comprising:
communicating the first set of data from the monitoring system to the mapping system; and
overlaying the first set of data on a display of the mapping system.

15. The method of claim 10, further comprising coalescing the first set of data and the second set of data to generate a consolidated report for recording and analysis.

16. A computer readable medium comprising one or more tangible media, wherein the one or more tangible media comprise:
code adapted to output in real-time a first set of data and a second set of data for display on a single display unit of an imaging system, wherein the imaging system comprises at least a monitoring system and a mapping system.

17. The computer readable medium, as recited in claim 16, wherein the code adapted to output in real-time the first set of data and the second set of data comprises code adapted to respond to a trigger.

18. The computer readable medium, as recited in claim 16, further comprising:
code adapted to communicate the second set of data from the mapping system to the monitoring system; and
code adapted to overlay the second set of data on a display of the monitoring system.

19. The computer readable medium, as recited in claim 16, further comprising:
code adapted to communicate the first set of data from the monitoring system to the mapping system; and
code adapted to overlay the first set of data on a display of the mapping system.

20. The computer readable medium, as recited in claim 16, further comprising code adapted to coalesce the first set of data and the second set of data to generate a consolidated report for recording and analysis.

21. A system for imaging, comprising:
a monitoring system;
a mapping system; and
a communication module operationally coupled to the monitoring system and the mapping system, wherein the communication module comprises a communication interface configured to facilitate bidirectional communication of data between the monitoring system and the mapping system.

22. The system of claim 21, wherein the communication interface is configured to facilitate real-time centralized data management.

23. The system of claim 22, wherein the communication interface is configured to:
communicate mapping data from the mapping system to the monitoring system; and
overlay the mapping data on a display of the monitoring system.

24. The system of claim 22, wherein the communication interface is configured to:
communicate physiological data from the monitoring system to the mapping system; and
overlay the physiological data on a display of the mapping system.

25. The system of claim 21, wherein the communication interface is further configured to:
coalesce data from the monitoring system and the mapping system to generate a consolidated report for display on the monitoring system, the mapping system, or both.
